SEQUENCE LISTING (29 sequences)

SEQ ID NO: 1 - protein, 209 aa, Solanum lycopersicum
MEFPSTPFDNSNNSEEREVGRRTDKRKQIDGEVKEYKSKNLKAERNRRQKLSERLLQLRSLVPNITNMTKETIITDAITYIRELQMNVDNLSEQLLEMEATQGEELETKNEEIIDTADEMGKWGIEPEVQVANIGPTKLWIKIVCQKKRGGLTKLMEAMNALGFDINDTSATASKGAILITSSVEVVRGGLTEANRIREILLEIIHGIY

SEQ ID NO: 2 - DNA, 2696 bp, Solanum lycopersicum
tgtaaaaaagaaaaagtgaaaggagaataaaatttctacttaaagcagctctggtcactcatttgtactttacaatgccagtaatactagtaacaaccacaaagtagtcacaaacataactagcaagaacattacccacttctattcttctttctgtttgattccttttctttcgggtggttgttactctttaaccctatagccttttacgagtcctcataacggaggtgggttttattaagttgacaactaggcgtaaataatcaaaccatgaggattccaactattatgcatactagttggttggcgatgtctatattcaatcattgttatgctatatgcatactagttagtatgtaaacataaaaaaagtacttgttcaacttagtcccaaacaagttaaactcggggtatatgaatatttactgaccatatttcccgtttgaatttatctctttcgggataacaataacagtgaaaagtgtaattagaccaaaactgaaaagggttgtagtgtctgaacctacaaaaaaaaactctacacactctctctttctcatgccaaaaatgagtccttccaagacaccccaagtacattgtgatcaattaggaaatgcccaacaaactacaacaagaagattcccacaaccaagaaacgtttctgttgtgaggagcgcacctatataaacctattctctgcctacaaaaaaaaaaacccagtgcagtactgcctcttcttttagctgcactcaacatattaatcattagcaccaatactctccatctatagatttcctttctaatttaggaattcattttaacatatttataacaatattctcttttaaatggtagtaattaggtgtgctaaaatggaattccccagtaccccatttgataattcaaacaactctgaagaaagggaagtaggaagaagaacagataaaaggaagcaaattgatggtgaagttaaagaatacaaatccaagaaccttaaggctgagagaaataggcgtcaaaaacttagcgaaaggcttcttcaattacgctcattggtcccaaacataacaaatatgacaaaagaaaccataatcactgacgccatcacctacattagggagctacaaatgaatgtggacaacctaagtgagcagcttcttgaaatggaagcaactcagggggaggaactggagacaaaaaatgaagagattatcgatactgcagacgagatgggtaaatggggcatagagcctgaagttcaagtggctaacattggcccaactaagctttggataaaaatagtctgccaaaagaaaagaggtggattaactaaactgatggaggcaatgaatgctcttggatttgatataaatgacaccagtgccactgcctctaaaggagctattcttattacttcatctgtggaggtggttagaggtggactaactgaagctaatcgaatcagagagatcttactggagatcatccacggaatctactagaaccagcatgaaaatcccctaaatcttaatagctttagtctcattggagaaatgccagagtacctctaatttttagttatggtgtcaaaaaaaggattcaatcatgttttcaaaatattgtatccaaattgtctgaaaaaagctagtgtgaactcttgtttttgctttcaaatattgatgaaacaacttatcatatgttactaattactctctcctatcttaattctatagcatttcatatgcagtcaagtttggcttaattcaactgaggcaaactttgaaatagaatatactactttttaacttggattttccatggtgggatccaggctccaaattttttggcttctctatatgaaacaatgaaaaaataacatttttggtcccatatccagctgaagaaacagctaaataataaaagccagcacattaagtgtcctttctgtttgaatattaaacaaacaccaatattagacgtgtgaagagtgaaatttatttctacctataatacaacaggacatttgtttcactgtcattaaattcttgttttcttctctgttctttgaccaatgtcgtcctcaatgaagagtcgaaaataagcctgccttaagcatcaccttcccttgtacaggatccaaataataaaagactacttggaaaaccaaaatcagacgtcgaatcgtcaaaatttaataccatggaaagtgtacctacccttcttataatattaacacgaaaaagactctcacaagtctatattaataggactaaaacatgcacacaattgtagcaatattcatattttctatggctaggcttagttttctcctcatttgcctagttatagctcaggttaacttgcttatgcgaaatgctaatgcacttagaaaactagacaccttcacagtagtcccaagtcctgcgccgtttgaaggaggaatggacagaggaagaagtgatcaagctccagcaatcacacatataaggactcatcattctttcaacaagttcatggctggtggagacttgatccttggcggcttcgccactgctctaattgcttccattttttgttacattcgagtcacaggacgaagaaaccaacatagtctgggctgaggcacagcatttttaattttgtactacttttagtttccaaatcaaatatatacacagggttcatgattttgctca

SEQ ID NO: 3 - protein, 230 aa, Solanum lycopersicum
MVVKVYGSAMAACPQRVMVCLIELGVDYELIHVDLDSLQQKKPDFLLLQPFGQVPVIEEGDFRLFESRAIIRYYAAKYEDKGKKLTGTTLEEKALVDQWLEVESNNYNDLVYNMVLQLLVFPKMGHKSDLIVVQKCANNLEKVFDIYEQRLSKSKYLAGDFFSLADLSHLPSLRFLMNEGGFAHLVTQRKYLHDWYLDISSRPSWSKVLDFMNLKKLEMLPGPPKEEVKV

SEQ ID NO: 4 - DNA, 960 bp, Solanum lycopersicum
tatatatatggtcaattattgatcacaaataagaagattattagaaatttcaacctggtgtactaggacatcacaacaaaaaaaaaaagaggaaaaatggtagtgaaagtgtatggttcagcaatggctgcatgtccacaaagggtcatggtttgtcttatagaattgggagtcgattatgaacttatacatgttgatcttgattctctccagcagaaaaaacctgatttcttgcttttacagccatttggacaggttcctgtcattgaagagggcgatttcaggcttttcgaatctagagcaataataaggtactatgcagcaaaatatgaagacaagggaaagaaactaaccggaacgacattggaagaaaaagctctagtagatcaatggctagaagtggaatccaacaactacaatgacttggtatacaacatggtactccaactcctcgtattccctaaaatgggacacaaaagtgacttgatcgtcgtacaaaaatgtgccaacaatttagagaaagtgttcgatatctatgaacaaaggttgtccaagagtaaatacttagcaggagattttttctccttagctgatctaagccacctccctagccttagatttttgatgaatgaaggtggctttgcacatttggtgactcaaaggaagtatttgcatgattggtatttggatatttcaagtaggccttcttggagcaaagtgttggacttcatgaatttgaagaaattagagatgttacccggcccacctaaagaagaagtaaaagtttaacaaacactacaacgccatgattattctgttacgagtggcatggatactgagaattcatattgccaactctgtctatctaatcagttaggttttgaaattgaagcgttttgtgtttcgttgtgttgtaatcaccaaaaataaaataaaattgaactatgggtttaccatcaa

SEQ ID NO: 5 - DNA, 23 bp, Artificial sequence
  Synthetic sequence
ggcgtcaaaaacttagcgaaagg

SEQ ID NO: 6 - DNA, 23 bp, Artificial sequence
  Synthetic sequence
atacaaatccaagaaccttaagg

SEQ ID NO: 7 - DNA, 23 bp, Artificial sequence
  Synthetic sequence
gaaagtgtatggttcagcaatgg

SEQ ID NO: 8 - DNA, 23 bp, Artificial sequence
  Synthetic sequence
caatggctgcatgtccacaaagg

SEQ ID NO: 9 - DNA, 20 bp, Artificial sequence
  Synthetic sequence
tggttgctgctcatcttcac

SEQ ID NO: 10 - DNA, 20 bp, Artificial sequence
  Synthetic sequence
gcaaagccaccttcattcat

SEQ ID NO: 11 - DNA, 20 bp, Artificial sequence
  Synthetic sequence
taggggattttcatgctggt

SEQ ID NO: 12 - DNA, 20 bp, Artificial sequence
  Synthetic sequence
gccaaaaatgagtccttcca

SEQ ID NO: 13 - DNA, 26 bp, Artificial sequence
  Synthetic sequence
actgtccctatctatgaaggttatgc

SEQ ID NO: 14 - DNA, 23 bp, Artificial sequence
  Synthetic sequence
gaaacagacaggacactcgcact

SEQ ID NO: 15 - DNA, 23 bp, Artificial sequence
  Synthetic sequence
aactctgaagaaagggaagtagg

SEQ ID NO: 16 - DNA, 23 bp, Artificial sequence
  Synthetic sequence
attcaaacaactctgaagaaagg

SEQ ID NO: 17 - DNA, 23 bp, Artificial sequence
  Synthetic sequence
aatggctgcatgtccacaaaggg

SEQ ID NO: 18 - DNA, 23 bp, Artificial sequence
  Synthetic sequence
atggtttgtcttatagaattggg

SEQ ID NO: 19 - DNA, 91 bp, Solanum lycopersicum
ccaagaaccttaaggctgagagaaataggcgtcaaaaacttagccaatggctgcatgtccacaaagggtcatggtttgtcttatagaattg

SEQ ID NO: 20 - DNA, 44 bp, Solanum lycopersicum
ccaagaaccttaaggctgagagaaataggcgtcaaaaacttagc

SEQ ID NO: 21 - DNA, 47 bp, Solanum lycopersicum
caatggctgcatgtccacaaagggtcatggtttgtcttatagaattg

SEQ ID NO: 22 - DNA, 89 bp, Solanum lycopersicum
cttcaccatcaatttgcttccttttatctgttcttcttcctactgtggacatgcagccattgctgaaccatacactttcactaccattt

SEQ ID NO: 23 - DNA, 44 bp, Solanum lycopersicum
cttcaccatcaatttgcttccttttatctgttcttcttcctact

SEQ ID NO: 24 - DNA, 45 bp, Solanum lycopersicum
gtggacatgcagccattgctgaaccatacactttcactaccattt

SEQ ID NO: 25 - DNA, 23 bp, Artificial sequence
  Synthetic sequence
cctacttccctttcttcagagtt

SEQ ID NO: 26 - DNA, 23 bp, Artificial sequence
  Synthetic sequence
ccctttgtggacatgcagccatt

SEQ ID NO: 27 - DNA, 86 bp, Solanum lycopersicum
gaattccccagtaccccatttgataattcaaacaactctgaattgggagtcgattatgaacttatacatgttgatcttgattctct

SEQ ID NO: 28 - DNA, 42 bp, Solanum lycopersicum
gaattccccagtaccccatttgataattcaaacaactctgaa

SEQ ID NO: 29 - DNA, 44 bp, Solanum lycopersicum
ttgggagtcgattatgaacttatacatgttgatcttgattctct

(19) United States
(12) Patent Application Publication
(10) Pub. No.: US 2023/0183725 A1
(43) Pub. Date: Jun. 15, 2023

(54) METHOD FOR OBTAINING MUTANT PLANTS BY TARGETED MUTAGENESIS

(71) Applicant: NUNHEMS B.V., Nunhem (NL)

(72) Inventor: Wim VRIEZEN, Nunhem (NL)

(21) Appl. No.: 17/924,249

(22) PCT Filed: May 7, 2021
(86) PCT No.: PCT/EP2021/062116

§ 371 Date: Nov. 9, 2022

(30) Foreign Application Priority Data
May 13, 2020 (EP) 20174401.8

(51) Int. Cl. C12N 15/82

(52) U.S. Cl. CPC C12N 15/8213

(57) ABSTRACT

The present invention relates to a method for the introduction and selection of a specific heritable mutation in a plant comprising transfecting plant cells with exogenous DNA, wherein said exogenous DNA encodes an RNA-guided DNA endonuclease, guide RNA suitable for directing the RNA-guided DNA endonuclease to induce said specific heritable mutation and a selection marker; regenerating plants from transfected cells to provide a plurality of T0 plants crossing the T0 plants with isogenic plants not comprising said exogenous DNA to provide a plurality of progeny plants; and selecting one or more plants having the heritable mutation from the progeny plants.

### FIELD OF THE INVENTION

The present invention relates to the field of plant breeding. Provided is a method for the introduction and selection of a specific heritable mutation in a plant comprising transfecting plant cells with exogenous DNA, wherein said exogenous DNA encodes an RNA-guided DNA endonuclease, guide RNA suitable for directing the RNA-guided DNA endonuclease to induce said specific heritable mutation and a selection marker; regenerating plants from transfected cells to provide a plurality of T0 plants crossing the T0 plants with isogenic plants not comprising said exogenous DNA to provide a plurality of progeny plants; and selecting one or more plants having the heritable mutation from the progeny plants.

### BACKGROUND

Mutagenesis techniques allow the creation of additional genetic diversity that is useful in plant breeding. The creation of such additional genetic diversity is particularly important since in most crop plants the genetic diversity available in the germplasm pool is limited due to the genetic bottleneck effect resulting from the domestication and subsequent selective breeding process. Induced mutagenesis methods, such as chemical mutagenesis and radiation mutagenesis accordingly are used in plant breeding for many decades in the search for new and improved plant traits useful in plant breeding.

The development of targeted mutagenesis methods in combination with the quickly developing knowledge of the genetic basis of traits in crop plants has opened further possibilities for creating new alleles that are useful in plant breeding. One particularly promising targeted mutagenesis method is genome editing using engineered nucleases, wherein a site-specific double-strand break is induced in the genomic DNA of a target cell using an engineered nuclease. Engineered nucleases useful in genome editing methods include meganucleases, zinc finger nucleases (ZFNs), transcription activator-like effector-based nucleases (TALEN), and clustered regularly interspaced short palindromic repeats (CRISPR)-associated nucleases. Particularly useful genome editing methods include CRISPR/Cas9-based targeted mutagenesis methods and CRISPR/Cpf1-based targeted mutagenesis methods; see e.g. Brooks et al. (2014) Plant Physiol 166, 1292-1297 and WO2016/205711 A1.

The double strand break induced by the engineered nuclease may be repaired by the cell's endogenous DNA double stranded break repair mechanisms, such as the homology directed repair mechanism (HDR) or by non-homologous end joining (NHEJ). Particularly HDR in the presence of a repair template, which is a DNA molecule used as a template in the host cell's DNA repair process, allows for a relatively efficient site-specific introduction of specific mutations of the genomic DNA in the target cell. By introducing a uniquely designed repair template into the target cell, a specific deletion, modification, insertion or replacement of DNA in the target cell can be achieved with a good efficiency and reliability. DNA repair of a double-stranded break by NHEJ is less predictable and may lead to different heritable mutations, such as the replacement and/or deletion and/or the insertion of one or more nucleotides at the site of the double-stranded break.

Certain mutations that may occur after inducing one or more double stranded breaks in the genomic DNA of a target plant cell, however, can be expected to only occur in a relatively low frequency. This is particularly the case if a very specific modification is desired such as very specific nucleotide deletion, substitution or insertion. Other modifications that can occur with low frequency is deletion of larger DNA fragments, inversion if DNA fragments or translocations. If such a low-frequency mutation represents the desired mutation, a plurality of mutant plants needs to be generated and screened in order identify and select a mutant plant comprising the specifically desired mutation. Particularly when the desired mutation only occurs at a very low frequency, a very high number of mutant plants need to be generated for screening, which is costly and time-consuming.

There is thus a need for a method for the introduction and selection of specific heritable mutations in plants which allows a more time- and cost-efficient screening of the plants having the desired heritable mutation.

### SUMMARY OF THE INVENTION

The present invention provides a method for the introduction and selection of a specific heritable mutation in a plant comprising: (a) transfecting plant cells with exogenous DNA, wherein said exogenous DNA encodes an RNA-guided DNA endonuclease, guide RNA (gRNA) suitable for directing the RNA-guided DNA endonuclease to induce said specific heritable mutation and a selection marker; (b) regenerating plants from transfected cells to provide a plurality of T0 plants; (c) crossing the T0 plants with isogenic plants not comprising said exogenous DNA to provide a plurality of progeny plants; and (d) selecting one or more plants having the heritable mutation from the progeny plants.

### BRIEF DESCRIPTION OF THE FIGURES

FIG. 1: Overview of the approach of Example 1. A. Schematic picture of the positions of the involved genes on Ch02 of tomato. MS refers the male sterility gene, and AA to the anthocyanin absence gene. The sizes and positions of the genes are not drawn to scale. B. The positions of the CRISPR-Cas induced double strand breaks in these two genes are shown as lighting flashes. A small proportion of the excised chromosomal fragments are repaired in the opposite orientation, leading to an induced inversion. Moreover, both genes (MS and AA) are knocked out. This leads to male sterility and anthocyanin absence. In hybrids the genetic distance between ms and aa is reduced to 0 cM, because of recombination suppression by the induced inversion. C. The primers for checking presence of induced inversions are shown as small arrows.

FIG. 2: Representation of a targeted induced inversion. The used CRISPR-Cas9 constructs contained two gRNAs per construct, such as gMS1 and gAA1, targeting the MS-gene and the AA-gene, respectively. When a double-strand break was induced at both sites in the same chromosome, inversion of the DNA fragment in between could occur. An inversion will lead to the inactivation of both genes since part of one gene is inversely fused to the remaining part of the other gene. Well-designed PCR-primers were used to unambiguously detect inversion-events. In this example, two primer-sites on the same DNA strand at the borders of the inversion (MS-R and AA-R) are oriented in the wild type genome in a manner that prevents any amplification in a PCR. However, after inversion the new locations of the primer binding sites are close together and in opposite directions, which makes amplification of a DNA fragment possible.

FIG. 3: The upper part of the figures represents the wild type reference genome. After inversion, the sequence at the gMS side is inverted and linked to the gAA side with is depicted by the arrows. The gRNA sequences, which were ˜1.1 Mbp apart on the reference genome, were linked together as shown by the sequence at the bottom. The alignment in the lower part of the figure shows that the DNA has been cleaved at the predicted location of the gRNA binding sites. The fraction of the gRNA sequence in bold corresponds to the sequence at the other side of the inversion. DNA sequence analysis of one end of an induced inversion after transfection with construct 1. The Sanger sequence is part of the PCR product generated with primers MS-R and AA-R and genomic DNA from protoplasts transfected with construct 1 containing the gRNA sequences gMS1 and gAA1. The Sanger sequence refers to the right-hand side of the inversion, and the flanking DNA at that side.

FIG. 4: The upper part of the figures represents the wild type reference genome. After inversion, the sequence at the gMS side is inverted and linked to the gAA side with is depicted by the arrows. The gRNA sequences, which were ˜1.1 Mbp apart on the reference genome, were linked together as shown by the sequence at the bottom. The alignment in the lower part of the figure shows that the DNA has been cleaved at the predicted location of the gRNA binding sites. The fraction of the gRNA sequence in bold corresponds to the sequence at the other side of the inversion. DNA sequence analysis of one end of an induced inversion after transfection with construct 3. The Sanger sequence is part of the PCR product generated with primers MS-F and AA-F and genomic DNA from protoplasts transfected with construct 3 containing the gRNA sequences gMS3 and gAA3. The Sanger sequence refers to the left-hand side of the inversion, and the flanking DNA at that side.

FIG. 5: The upper part of the figures represents the wild type reference genome. After inversion, the sequence at the gMS side is inverted and linked to the gAA side with is depicted by the arrows. The gRNA sequences, which were ˜1.1 Mbp apart on the reference genome, were linked together as shown by the sequence at the bottom. The alignment in the lower part of the figure shows that the DNA has been cleaved at the predicted location of the gRNA binding sites. The fraction of the gRNA sequence in bold corresponds to the sequence at the other side of the inversion. DNA sequence analysis of one end of an induced inversion after transfection with construct 4. The Sanger sequence is part of the PCR product generated with primers MS-R and AA-R and genomic DNA from protoplasts transfected with construct 4 containing the gRNA sequences gMS4 and gAA4. The Sanger sequence refers to the right-hand side of the inversion, and the flanking DNA at that side.

### DETAILED DESCRIPTION OF THE INVENTION

### General Definitions

It is to be understood that this invention is not limited to the particular methodology or protocols. It is also to be understood that the terminology used herein is for the purpose of describing particular embodiments only, and is not intended to limit the scope of the present invention which will be limited only by the appended claims. It must be noted that as used herein and in the appended claims, the singular forms “a,” “and,” and “the” include plural reference unless the context clearly dictates otherwise. Thus, for example, reference to “a vector” is a reference to one or more vectors and includes equivalents thereof known to those skilled in the art, and so forth. The term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent, preferably 10 percent up or down (higher or lower). As used herein, the word or means any one member of a particular list and also includes any combination of members of that list. The words “comprise,” “comprising,” “include,” “including,” and “includes” when used in this specification and in the following claims are intended to specify the presence of one or more stated features, integers, components, or steps, but they do not preclude the presence or addition of one or more other features, integers, components, steps, or groups thereof. For clarity, certain terms used in the specification are defined and used as follows:

The term “genome” relates to the genetic material of an organism. It consists of DNA. The genome includes both the genes and the non-coding sequences of the DNA.

The term “gene” means a (genomic) DNA sequence comprising a region (transcribed region), which is transcribed into a messenger RNA molecule (mRNA) in a cell, and an operably linked (also described herein as regulatory sequence, e.g. a promoter). A gene may thus comprise several operably linked sequences, such as a promoter, a 5′ leader sequence comprising e.g. sequences involved in translation initiation, a (protein) coding region (cDNA or genomic DNA) and a 3′ non-translated sequence comprising e.g. transcription termination sites. Different alleles of a gene are thus different alternative forms of the gene, which may be in the form of e.g. differences in one or more nucleotides of the genomic DNA sequence (e.g. in the promoter sequence, the exon sequences, intron sequences, etc.), mRNA and/or amino acid sequence of the encoded protein. A gene may be endogenous gene, which is defined herein as a genetic locus comprising a DNA sequence originating from the species of origin.

An induced genetic modification to a plant may be a cisgenic modification, which in the context of the present invention refers to a modification of a plant genome via the integration of whole (intact) genes, including coding sequences and their native regulatory elements (e.g. promotors and terminators), derived from the same or a sexually compatible species. An induced genetic modification to a plant may also be an intragenic modification, which in the context of the present invention refers to modifications of a plant genome via the integration of recombinant genes, including coding sequences and their regulatory elements (e.g. promotors and terminators), derived from the same or a sexually-compatible species. Intragenesis differs from cisgenesis in that different combinations of genetic elements may be used, e.g. a promoter from a different gene. A plant into which any DNA sequences from a non-sexually compatible organism or organism from a different genus have been integrated into the genome is referred to as a “transgenic plant”. “Crossable species” include the species within the taxonomic family of the organism. “Non-crossable species” are these outside of the taxonomic family of the species.

The “promoter” of a gene sequence is defined as a region of DNA that initiates transcription of a particular gene. Promoters are located near the genes they transcribe, on the same strand and upstream on the DNA. Promoters can be about 100-1000 base pairs long. In one aspect the promoter is defined as the region of about 1000 base pairs or more e.g. about 1500 or 2000, upstream of the start codon (i.e. ATG) of the protein encoded by the gene.

“Expression of a gene” refers to the process wherein a DNA region, which is operably linked to appropriate regulatory regions, particularly a promoter, is transcribed into an RNA, which is biologically active, i.e. which is capable of being translated into a biologically active protein or peptide (or active peptide fragment) or which is active itself (e.g. in posttranscriptional gene silencing or RNAi). The coding sequence may be in sense-orientation and encodes a desired, biologically active protein or peptide, or an active peptide fragment.

A “quantitative trait locus”, or “QTL” is a chromosomal locus that encodes for one or more alleles that affect the expressivity of a continuously distributed (quantitative) phenotype.

“Physical distance” between loci (e.g. between genes and/or between molecular markers and/or between phenotypic markers) on the same chromosome is the actual physical distance expressed in bases or base pairs (bp), kilo bases or kilo base pairs (kb) or megabases or mega base pairs (Mb).

“Genetic distance” between loci (e.g. between molecular markers and/or between phenotypic markers) on the same chromosome is measured by frequency of crossing-over, or recombination frequency (RF) and is indicated in centimorgans (cM). One cM corresponds to a recombination frequency of 1%. If no recombinants can be found, the RF is zero and the loci are either extremely close together physically or they are identical. The further apart two loci are, the higher the RF.

The term “meiotic recombination” refers to the genetic recombination involving the pairing of homologous chromosomes that occurs in eukaryotes during meiosis. The pairing of homologous chromosomes may be followed by information transfer between said chromosomes. This information transfer may occur without physical exchange (a section of genetic material is copied from one chromosome to another, without the donating chromosome being changed) or by the breaking and re-joining of DNA strands, which forms a newly recombined DNA molecule.

The term “RNA-guided DNA endonuclease” refers to an enzyme capable of cleaving the phosphodiester bond within a target nucleic acid (e.g. target DNA), whereby the cleavage site is specifically determined by a guide RNA that is associated to the RNA-guided DNA endonuclease to form a complex. Such RNA-guided DNA endonuclease are typically associated with the Clustered Regularly Interspaced Short Palindromic Repeats (CRISPR) adaptive immune system in bacteria, wherein different RNA-guided DNA endonucleases are derived from different bacteria species. RNA-guided DNA endonucleases are well-known in the art including, but not limited to, Cas9, Cas12a, Cas12b, Cas12c, and Cas13; see e.g. Schindele 2018 doi:10.1002/1873-3468.13073.

The term “guide RNA” (gRNA) refers to a non-coding short RNA capable of associating to RNA-guided DNA endonuclease and which specifically binds to a target nucleic acid sequence, thereby directing the RNA-guided DNA endonuclease to said target nucleic acid. The gRNA comprises a “spacer nucleic acid”, which is a nucleic acid comprising a sufficient number of bases that are complementary to the target nucleic acid to specifically bind to the target nucleic acid, thereby capable of directing the RNA-guided DNA endonuclease to the region of interest of the target nucleic acid.

The term “CRISPR RNA” (crRNA) refers to a component of the gRNA, wherein the crRNA is the element of the gRNA comprises the spacer nucleic acid that locates the correct segment of host DNA. In the CRISPR-Cas9 system the crRNA comprises a region that binds to tracrRNA forming the active gRNA. The active gRNA comprised in the CRISPR-Cas9 system may comprise a separate crRNA and a separate tracrRNA forming a complex or may comprise an active sgRNA comprising a crRNA and a tracrRNA that are covalently connected, e.g. by a linker. The term “trans-activating crRNA” (tracrRNA) refers to a component of the gRNA comprised in the CRISPR-Cas9 system that generally comprises a hairpin loop and which binds to crRNA forming an active gRNA.

The term “selection marker” refers to a gene that, when introduced into a cell, confers a trait suitable for artificial selection to indicate the success of a transfection meant to introduce foreign DNA into a cell. Particularly suitable selection markers include antibiotic resistance genes such as ntp II (neomycin phosphotransferase II for kanamycin resistance), hpt (hygromycin phosphotransferase for hygromycin B resistance) and aad A (aminoglycoside-3 adenyltransferase for streptomycin resistance).

The term “transfection” or “transformation” or “transduction” relates to the process of deliberately introducing nucleic acids into cells.

“Wild type allele” (WT) refers herein to a version of a gene encoding a fully functional protein (wild type protein).

For example, the term “wild type MS10 allele” or “MS10 allele” or “wild type allele of the MS10 gene” refers to the fully functional allele of the MS10 gene, which allows normal protein function (i.e. normal protein expression in combination with normal enzymatic activity of the expressed protein) when compared to a wild type MS10 allele. The MS10 gene encodes a basic helix-loop-helix transcription factor. One example of a wild type MS10 allele in the species Solanum lycopersicum for instance is the wild type genomic DNA which encodes the wild type MS10 cDNA (mRNA) sequence depicted in SEQ ID NO:2. The protein sequence encoded by this wild type MS10 cDNA has 209 amino acid residues and is depicted in SEQ ID NO:1, which corresponds to NCBI reference sequence XM_026029418.1. The wild type Solanum lycopersicum MS10 allele further comprises functional variants of the wild type genomic DNA which encodes the wild type MS10 cDNA and amino acid sequences as described herein. Whether a certain variant of the herein specifically described wild type MS10 allele represents a “functional variant” can be determined by using routine methods, including, but not limited to phenotypic testing for normal viable pollen production and in silico prediction of amino acid changes that affect protein function. For instance, a web-based computer program SIFT (Sorting Intolerant from Tolerant) is a program that predicts whether an amino acid substitution affects protein function; see world wide web at sift.bii.a-star.edu.sg/. Functionally important amino acids will be conserved in the protein family, and so changes at well-conserved positions tend to be predicted as not tolerated or deleterious; see also Ng and Henikoff (2003) Nucleic Acids Res 31(13): 3812-3814. For example, if a position in an alignment of a protein family only contains the amino acid isoleucine, it is presumed that substitution to any other amino acid is selected against and that isoleucine is necessary for protein function. Therefore, a change to any other amino acid will be predicted to be deleterious to protein function. If a position in an alignment contains the hydrophobic amino acids isoleucine, valine and leucine, then SIFT assumes, in effect, that this position can only contain amino acids with hydrophobic character. At this position, changes to other hydrophobic amino acids are usually predicted to be tolerated but changes to other residues (such as charged or polar) will be predicted to affect protein function. An alternative tool useful for the prediction of protein function is Provean; see world wide web at provean.jcvi.org/index.php. Also, an ortholog of the Solanum lycopersicum MS10 gene, particularly in a wild relative of the species Solanum lycopersicum, may be a functional variant of the wild type MS10 allele provided that said variant allows normal protein function.

As a further example, the “wild type AA allele” or “AA allele” or “wild type allele of the AA gene” refers to the fully functional allele of the AA gene, which allows normal protein function (i.e. normal protein expression in combination with normal enzymatic activity of the expressed protein) when compared to a wild type AA allele. The AA gene encodes a glutathione S-transferase enzyme. One example of a wild type AA allele in the species Solanum lycopersicum for instance is the wild type genomic DNA which encodes the wild type AA cDNA (mRNA) sequence depicted in SEQ ID NO:4. The protein sequence encoded by this wild type AA cDNA has 230 amino acid residues and is depicted in SEQ ID NO:3, which corresponds to NCBI reference sequence XM_004232621.4. The wild type Solanum lycopersicum AA allele further comprises functional variants of the wild type genomic DNA which encodes the wild type AA cDNA and amino acid sequences as described herein. Whether a certain variant of the herein specifically described wild type AA allele represents a “functional variant” can be determined by using routine methods, including, but not limited to testing of enzymatic activity, phenotypic testing for hypocotyl colour and in silico prediction of amino acid changes that affect protein function as further described herein above. Also, an ortholog of the Solanum lycopersicum AA gene, particularly in a wild relative of the species Solanum lycopersicum, may be a functional variant of the wild type AA allele provided that said variant allows normal protein function.

“Mutant allele” refers herein to an allele comprising one or more mutations when compared to the wild type allele, resulting in the trait of the present invention. The one or more mutations may be in the coding sequence (mRNA, cDNA or genomic sequence) or in the associated non-coding sequence and/or regulatory sequence regulating the level of expression of the coding sequence. Such mutation(s) (e.g. insertion, inversion, deletion and/or replacement of one or more nucleotide(s)) may lead to the encoded protein having reduced in vitro and/or in vivo functionality (reduced function) or no in vitro and/or in vivo functionality (loss-of-function), e.g. due to the protein being truncated or having an amino acid sequence wherein one or more amino acids are deleted, inserted or replaced. Such changes may lead to the protein having a different 3D conformation, being targeted to a different sub-cellular compartment, having one or more modified catalytic domains, having a modified binding activity to nucleic acids or proteins, etc. preferably, the mutant allele of the present invention encodes a truncated protein having decreased function or loss-of-function when compared to the wild type protein. Furthermore, the mutation(s) (e.g. insertion, inversion, deletion and/or replacement of one or more nucleotide(s)) may lead to the encoded protein having reduced expression or no protein expression.

For example, the term “mutant ms10 allele” or “ms10 allele” or “mutant allele of the MS10 gene” or “mutant allele of the wild type MS10 gene” inter alia refers to an allele of the MS10 gene comprising one or more mutations in the coding sequence, which one or more mutations leads to a reduced function or loss-of-function of encoded gene product and which causes the plants to have the male sterility trait when the mutant allele is in homozygous form. The term “male sterility” or “male sterility trait” refers to a plant trait which results in the failure of the plant to produce functional anthers, pollen, or male gametes. The term mutant ms10 allele also comprises knock-out ms10 alleles and knock-down ms10 alleles, as well as ms10 alleles encoding a mutant ms10 protein having reduced function or no function. As used herein, the term “knockout allele” refers to an allele wherein the expression of the respective (wild type) gene is not detectable anymore. A “knock-down” allele has reduced expression of the respective (wild type) gene compared to the wild type allele.

As a further example, the term “mutant aa allele” or “aa allele” or “mutant allele of the AA gene” or “mutant allele of the wild type AA gene” inter alia refers to an allele of the AA gene comprising one or more mutations in the coding sequence, which one or more mutations leads to a reduced function or loss-of-function of encoded gene product and which causes the plants to have the anthocyanin absent trait when the mutant allele is in homozygous form. The term “anthocyanin absent” or “anthocyanin absent trait” refers to a plant trait which results in the absence of anthocyanin coloration in the hypocotyls of said plant. The term mutant aa allele also comprises knock-out aa alleles and knock-down aa alleles, as well as aa alleles encoding a mutant aa protein having reduced function or no function.

The term “induced mutant allele” as used herein refers to any allele of the wild type gene resulting in the trait of the present invention which is produced by human intervention, such as mutagenesis. Preferably, the induced mutant allele cannot be found in plants in the natural population or breeding population.

The term “natural mutant allele” as used herein refers to any allele of the wild type gene resulting in the trait of the present invention wherein the mutant allele evolved without direct human intervention. Preferably, the natural mutant allele can be found in plants in the natural population or breeding population.

The term “orthologous gene” or “ortholog” is defined as genes in different species that have evolved through speciation events. It is generally assumed that orthologs have the same biological functions in different species. Accordingly, it is particularly preferred that the protein encoded by the ortholog of the wild type Solanum lycopersicum MS10 gene in wild relatives of the species Solanum lycopersicum has the same biological function as the wild type Solanum lycopersicum MS10 protein. Furthermore, it is particularly preferred that the protein encoded by the ortholog of the wild type Solanum lycopersicum AA gene in wild relatives of the species Solanum lycopersicum has the same biological function as the wild type Solanum lycopersicum AA protein. Methods for the identification of orthologs is very well known in the art as it accomplishes two goals: delineating the genealogy of genes to investigate the forces and mechanisms of evolutionary process and creating groups of genes with the same biological functions (Fang G, et al (2010) Getting Started in Gene Orthology and Functional Analysis. PLoS Comput Biol 6(3): e1000703. doi:10.1371/journal.pcbi.1000703). For instance, orthologs of a specific gene or protein can be identified using sequence alignment or sequence identity of the gene sequence of the protein of interest with gene sequences of other species. Gene alignments or gene sequence identity determinations can be done according to methods known in the art, e.g. by identifying nucleic acid or protein sequences in existing nucleic acid or protein database (e.g. GENBANK, SWISSPROT, TrEMBL) and using standard sequence analysis software, such as sequence similarity search tools (BLASTN, BLASTP, BLASTX, TBLAST, FASTA, etc.).

“Introgression fragment” or “introgression segment” or “introgression region” refers to a chromosome fragment (or chromosome part or region) which has been introduced into another plant of the same or related species by crossing or traditional breeding techniques, such as backcrossing, i.e. the introgressed fragment is the result of breeding methods referred to by the verb “to introgress” (such as backcrossing). It is understood that the term “introgression fragment” never includes a whole chromosome, but only a part of a chromosome. The introgression fragment can be large, e.g. even three-quarters or half of a chromosome, but is preferably smaller, such as about 15 Mb or less, such as about 10 Mb or less, about 9 Mb or less, about 8 Mb or less, about 7 Mb or less, about 6 Mb or less, about 5 Mb or less, about 4 Mb or less, about 3 Mb or less, about 2.5 Mb or 2 Mb or less, about 1 Mb (equals 1,000,000 base pairs) or less, or about 0.5 Mb (equals 500,000 base pairs) or less, such as about 200,000 bp (equals 200 kilo base pairs) or less, about 100,000 bp (100 kb) or less, about 50,000 bp (50 kb) or less, about 25,000 bp (25 kb) or less.

The term “isogenic plant” refers to two plants which are genetically identical except for the mutant allele of interest. The impact of the mutant allele of interest, for example on the phenotype of mutant plants, can then be compared between the plant line (variety) comprising the mutation and its isogenic line not comprising the mutation.

The term “nucleic acid sequence” or “nucleic acid molecule” or polynucleotide are used interchangeably and refer to a DNA or RNA molecule in single or double stranded form, particularly a DNA encoding a protein or protein fragment according to the invention. An “isolated nucleic acid sequence” refers to a nucleic acid sequence which is no longer in the natural environment from which it was isolated, e.g. the nucleic acid sequence in a bacterial host cell or in the plant nuclear or plastid genome.

The terms “protein”, “peptide sequence”, “amino acid sequence” or “polypeptide” are used interchangeably and refer to molecules consisting of a chain of amino acids, without reference to a specific mode of action, size, 3-dimensional structure or origin. A “fragment” or “portion” of a protein may thus still be referred to as a “protein”. An “isolated protein” is used to refer to a protein which is no longer in its natural environment, for example in vitro or in a recombinant bacterial or plant host cell.

An “active protein” or “functional protein” is a protein which has protein activity as measurable in vitro, e.g. by an in vitro activity assay, and/or in vivo, e.g. by the phenotype conferred by the protein. A “wild type” protein is a fully functional protein, as present in the wild type plant. A “mutant protein” is herein a protein comprising one or more mutations in the nucleic acid sequence encoding the protein, whereby the mutation results in (the mutant nucleic acid molecule encoding) a protein having altered activity, preferably a protein having reduced activity, most preferably a protein having no activity.

“Functional derivatives” of a protein as described herein are fragments, variants, analogues, or chemical derivatives of the protein which retain at least a portion of the activity or immunological cross reactivity with an antibody specific for the mutant protein.

A fragment of a mutant protein refers to any subset of the molecule.

Variant peptides may be made by direct chemical synthesis, for example, using methods well known in the art.

An analogue of a mutant protein refers to a non-natural protein substantially similar to either the entire protein or a fragment thereof.

A “mutation” in a nucleic acid molecule is a change of one or more nucleotides compared to the wild type sequence, e.g. by replacement, deletion or insertion of one or more nucleotides.

A “mutation” in an amino acid molecule making up a protein is a change of one or more amino acids compared to the wild type sequence, e.g. by replacement, deletion or insertion of one or more amino acids. Such a protein is then also referred to as a “mutant protein”.

A “point mutation” is the replacement of a single nucleotide, or the insertion or deletion of a single nucleotide.

A “nonsense mutation” is a (point) mutation in a nucleic acid sequence encoding a protein, whereby a codon in a nucleic acid molecule is changed into a stop codon. This results in a pre-mature stop codon being present in the mRNA and results in translation of a truncated protein. A truncated protein may have decreased function or loss of function.

A “missense or non-synonymous mutation” is a (point) mutation in a nucleic acid sequence encoding a protein, whereby a codon is changed to code for a different amino acid. The resulting protein may have decreased function or loss of function.

A “splice-site mutation” is a mutation in a nucleic acid sequence encoding a protein, whereby RNA splicing of the pre-mRNA is changed, resulting in an mRNA having a different nucleotide sequence and a protein having a different amino acid sequence than the wild type. The resulting protein may have decreased function or loss of function.

A “frame shift mutation” is a mutation in a nucleic acid sequence encoding a protein by which the reading frame of the mRNA is changed, resulting in a different amino acid sequence. The resulting protein may have decreased function or loss of function.

A “deletion” in context of the invention shall mean that anywhere in a given nucleic acid sequence at least one nucleotide is missing compared to the nucleic sequence of the corresponding wild type sequence or anywhere in a given amino acid sequence at least one amino acid is missing compared to the amino acid sequence of the corresponding (wild type) sequence.

An “inversion” in context of the invention shall mean a mutation wherein in a given nucleic acid sequence the nucleotide sequence of a fragment of at least 3 or more nucleotides is reversed when compared to the wild type nucleotide sequence.

A “duplication” in context of the invention shall mean a mutation wherein the nucleotide sequence of a fragment of at least 3 or more nucleotides in a given nucleic acid sequence is duplicated when compared to the wild type nucleotide sequence. The duplicated fragment may comprise one or more whole genes. The duplicated fragment may further comprise one or more regulatory regions. Furthermore, the fragment may be duplicated within the same chromosome of the genome or may be duplicated on a different chromosome within the genome.

A “translocation” in context of the invention shall mean a mutation wherein in a given nucleic acid sequence the nucleotide sequence of a fragment of at least 3 or more nucleotides is deleted from one locus in the genome and inserted at a different locus in the genome when compared to the wild type nucleotide sequence. The translocated fragment of at least 3 or more nucleotides may comprise one or more whole genes. The translocated fragment of at least 3 or more nucleotides may further comprise one or more regulatory regions. Furthermore, the fragment may be translocated within the same chromosome of the genome or may be translocated on a different chromosome within the genome.

A “truncation” shall be understood to mean that at least one nucleotide at either the 3′-end or the 5′-end of the nucleotide sequence is missing compared to the nucleic sequence of the corresponding wild type sequence or that at least one amino acid at either the N-terminus or the C-terminus of the protein is missing compared to the amino acid sequence of the corresponding wild type protein, whereby in a 3′-end or C-terminal truncation at least the first nucleotide at the 5′-end or the first amino acid at the N-terminus, respectively, is still present and in a 5′-end or N-terminal truncation at least the last nucleotide at the 3′-end or the last amino acid at the C-terminus, respectively, is still present. The 5′-end is determined by the ATG codon used as start codon in translation of a corresponding wild type nucleic acid sequence.

“Replacement” shall mean that at least one nucleotide in a nucleic acid sequence or one amino acid in a protein sequence is different compared to the corresponding wild type nucleic acid sequence or the corresponding wild type amino acid sequence, respectively, due to an exchange of a nucleotide in the coding sequence of the respective protein.

“Insertion” shall mean that the nucleic acid sequence or the amino acid sequence of a protein comprises at least one additional nucleotide or amino acid compared to the corresponding wild type nucleic acid sequence or the corresponding wild type amino acid sequence, respectively.

“Pre-mature stop codon” in context with the present invention means that a stop codon is present in a coding sequence (cds) which is closer to the start codon at the 5′-end compared to the stop codon of a corresponding wild type coding sequence.

A “mutation in a regulatory sequence”, e.g. in a promoter or enhancer of a gene, is a change of one or more nucleotides compared to the wild type sequence, e.g. by replacement, deletion or insertion of one or more nucleotides, leading for example to decreased or no mRNA transcript of the gene being made. The “promoter of a gene sequence”, accordingly is defined as a region of DNA that initiates transcription of a particular gene. Promoters are located near the genes they transcribe, on the same strand and upstream on the DNA. Promoters can be about 100-1000 base pairs long. In one aspect, the promoter is defined as the region of about 2000 base pairs or more upstream of the start codon (i.e. ATG) of the protein encoded by the gene, preferably, the promoter is the region of about 1500 base pairs upstream of the start codon, more preferably the promoter is the region of about 1000 base pairs upstream of the start codon.

As used herein, the term “operably linked” refers to a linkage of polynucleotide elements in a functional relationship. A nucleic acid is “operably linked” when it is placed into a functional relationship with another nucleic acid sequence. For instance, a promoter, or rather a transcription regulatory sequence, is operably linked to a coding sequence if it affects the transcription of the coding sequence. Operably linked means that the nucleic acid sequences being linked are typically contiguous.

“Sequence identity” and “sequence similarity” can be determined by alignment of two peptide or two nucleotide sequences using global or local alignment algorithms. Sequences may then be referred to as “substantially identical” when they are optimally aligned by for example the programs GAP or BESTFIT or the Emboss program “Needle” (using default parameters, see below) share at least a certain minimal percentage of sequence identity (as defined further below). These programs use the Needleman and Wunsch global alignment algorithm to align two sequences over their entire length, maximizing the number of matches and minimizing the number of gaps. Generally, the default parameters are used, with a gap creation penalty=10 and gap extension penalty=0.5 (both for nucleotide and protein alignments). For nucleotides the default scoring matrix used is DNAFULL and for proteins the default scoring matrix is Blosum62 (Henikoff & Henikoff, 1992, PNAS 89, 10915-10919). Sequence alignments and scores for percentage sequence identity may for example be determined using computer programs, such as EMBOSS, (as available on the Internet by ebi.ac.uk at http://www.ebi.ac.uk under/Tools/psa/emboss_needle/). Alternatively, sequence similarity or identity may be determined by searching against databases such as FASTA, BLAST, etc., but hits should be retrieved and aligned pairwise to compare sequence identity. Two proteins or two protein domains, or two nucleic acid sequences have “substantial sequence identity” if the percentage sequence identity is at least 95%, 96%, 97%, 98%, 98.3%, 98.7%, 99.0%, or 99.3% or more preferably 99.7% (as determined by Emboss “needle” using default parameters, i.e. gap creation penalty=10, gap extension penalty=0.5, using scoring matrix DNAFULL for nucleic acids and Blosum62 for proteins). Such sequences are also referred to as ‘variants’ herein, e.g. other variants of alleles causing the male sterility trait of the present invention and/or the anthocyanin absent trait of the present invention and proteins than the specific nucleic acid and amino acid sequences disclosed herein can be identified, which have the same effect on male sterility and/or the absence of anthocyanin in the hypocotyl as the plants of the present invention.

The term “hybridisation” as used herein is generally used to mean hybridisation of nucleic acids at appropriate conditions of stringency (stringent hybridisation conditions) as would be readily evident to those skilled in the art depending upon the nature of the probe sequence and target sequences. Conditions of hybridisation and washing are well-known in the art, and the adjustment of conditions depending upon the desired stringency by varying incubation time, temperature and/or ionic strength of the solution are readily accomplished. See, for example, Sambrook, J. et al., Molecular Cloning: A Laboratory Manual, 2nd edition, Cold Spring Harbor Press, Cold Spring Harbor, N.Y., 1989. The choice of conditions is dictated by the length of the sequences being hybridised, in particular, the length of the probe sequence, the relative G-C content of the nucleic acids and the amount of mismatches to be permitted. Low stringency conditions are preferred when partial hybridisation between strands that have lesser degrees of complementarity is desired. When perfect or near perfect complementarity is desired, high stringency conditions are preferred. For typical high stringency conditions, the hybridisation solution contains 6×S.S.C., 0.01 M EDTA, 1×Denhardt's solution and 0.5% SOS. hybridisation is carried out at about 68° C. for about 3 to 4 hours for fragments of cloned DNA and for about 12 to about 16 hours for total eukaryotic DNA. For lower stringencies the temperature of hybridisation is reduced to about 42° C. below the melting temperature (T_{M}) of the duplex. The T_{M} is known to be a function of the G-C content and duplex length as well as the ionic strength of the solution.

As used herein, the phrase “hybridizes” to a DNA or RNA molecule means that the molecule that hybridizes, e.g., oligonucleotide, polynucleotide, or any nucleotide sequence (in sense or antisense orientation) recognizes and hybridizes to a sequence in another nucleic acid molecule that is of approximately the same size and has enough sequence similarity thereto to effect hybridisation under appropriate conditions. For example, a 100 nucleotide long molecule from the 3′ coding or non-coding region of a gene will recognize and hybridize to an approximately 100 nucleotide portion of a nucleotide sequence within the 3′ coding or non-coding region of that gene or any other plant gene so long as there is about 70% or more sequence similarity between the two sequences. It is to be understood that the size of the corresponding portion will allow for some mismatches in hybridisation such that the corresponding portion may be smaller or larger than the molecule which hybridizes to it, for example 20-30% larger or smaller, preferably no more than about 12-15% larger or smaller.

As used herein, the phrase “a sequence comprising at least 95% sequence identity” or “a sequence comprising at least 95% amino acid sequence identity” or “a sequence comprising at least 95% nucleotide sequence identity” means a sequence having at least 95% e.g. at least 96%, 97%, 98%, 98.3%, 98.7%, 99.0%, or 99.3% or more preferably 99.7% sequence identity when compared with the reference sequence that is indicated. Sequence identity can be determined according the methods described herein.

A “fragment” of the gene or DNA sequence refers to any subset of the molecule, e.g., a shorter polynucleotide or oligonucleotide. In one aspect the fragment comprises the mutation as defined by the invention.

A “variant” of the gene or DNA refers to a molecule substantially similar to either the entire gene or a fragment thereof, such as a nucleotide substitution variant having one or more substituted nucleotides, but which maintains the ability to hybridize with the particular gene or to encode mRNA transcript which hybridizes with the native DNA. Preferably the variant comprises the mutant allele as defined by the invention.

As used herein, the term “plant” includes the whole plant or any parts or derivatives thereof, such as plant organs (e.g., harvested or non-harvested flowers, leaves, etc.), plant cells, plant protoplasts, plant cell or tissue cultures from which whole plants can be regenerated, regenerable or non-regenerable plant cells, plant calli, plant cell clumps, and plant cells that are intact in plants, or parts of plants, such as embryos, pollen, ovules, ovaries (e.g., harvested tissues or organs), flowers, leaves, seeds, tubers, clonally propagated plants, roots, stems, cotyledons, hypocotyls, root tips and the like. Also, any developmental stage is included, such as seedlings, immature and mature, etc.

A “plant line” or “breeding line” refers to a plant and its progeny. As used herein, the term “inbred line” refers to a plant line which has been repeatedly selfed and is nearly homozygous for every characteristic. Thus, an “inbred line” or “parent line” refers to a plant which has undergone several generations (e.g. at least 5, 6, 7 or more) of inbreeding, resulting in a plant line with a high uniformity.

“Plant variety” is a group of plants within the same botanical taxon of the lowest grade known, which (irrespective of whether the conditions for the recognition of plant breeders rights are fulfilled or not) can be defined on the basis of the expression of characteristics that result from a certain genotype or a combination of genotypes, can be distinguished from any other group of plants by the expression of at least one of those characteristics, and can be regarded as an entity, because it can be multiplied without any change. Therefore, the term “plant variety” cannot be used to denote a group of plants, even if they are of the same kind, if they are all characterized by the presence of 1 locus or gene (or a series of phenotypical characteristics due to this single locus or gene), but which can otherwise differ from one another enormously as regards the other loci or genes. “F1, F2, etc.” refers to the consecutive related generations following a cross between two parent plants or parent lines. The plants grown from the seeds produced by crossing two plants or lines is called the F1 generation. Selfing the F1 plants results in the F2 generation, etc. “F1 hybrid” plant (or F1 seed, or hybrid) is the generation obtained from crossing two inbred parent lines. “Selfing”, accordingly, refers to the self-pollination of a plant, i.e. to the union of gametes from the same plant.

“Backcrossing” refers to a breeding method by which a (single) trait, such as the male sterility trait and/or the anthocyanin absent trait, can be transferred from one genetic background (also referred to as “donor” generally, but not necessarily, this is an inferior genetic background) into another genetic background (also referred to as “recurrent parent”; generally, but not necessarily, this is a superior genetic background). An offspring of a cross (e.g. an F1 plant obtained by crossing a first plant of a certain plant species comprising the mutant allele of the present invention with a second plant of the same plant species or of a different plant species that can be crossed with said first plant species wherein said second plant species does not comprise the mutant allele of the present invention; or an F2 plant or F3 plant, etc., obtained by selfing the F1) is “backcrossed” to a parent plant of said second plant species. After repeated backcrossing, the trait of the donor genetic background, e.g. the mutant allele conferring the male sterility trait and/or the anthocyanin absent trait as described herein, will have been incorporated into the recurrent genetic background. The terms “gene converted” or “conversion plant” or “single locus conversion” in this context refer to plants which are developed by backcrossing wherein essentially all of the desired morphological and/or physiological characteristics of the recurrent parent are recovered in addition to the one or more genes transferred from the donor parent. The plants grown from the seeds produced by backcrossing of the F1 plants with the second parent plant line is referred to as the “BC1 generation”. Plants from the BC1 population may be selfed resulting in the BC1F2 generation or backcrossed again with the cultivated parent plant line to provide the BC2 generation. An “M1 population” is a plurality of mutagenized seeds/plants of a certain plant line. “M2, M3, M4, etc.” refers to the consecutive generations obtained following selfing of a first mutagenized seed/plant (M1). The term “T0 plant” as used herein relates to a plant regenerated from one or more transfected cells. “T1, T2, T3, etc.” refers to the consecutive generations obtained following selfing of a first transfected seed/plant (T0).

The term “cultivated plant” or “cultivar” refers to plants of a given species, e.g. varieties, breeding lines or cultivars of the said species, cultivated by humans and having good agronomic characteristics. The so-called heirloom varieties or cultivars, i.e. open pollinated varieties or cultivars commonly grown during earlier periods in human history and often adapted to specific geographic regions, are in one aspect of the invention encompassed herein as cultivated plants. The term “cultivated plant” does not encompass wild plants. “Wild plants” include for example wild accessions.

The term “food” is any substance consumed to provide nutritional support for the body. It is usually of plant or animal origin, and contains essential nutrients, such as carbohydrates, fats, proteins, vitamins, or minerals. The substance is ingested by an organism and assimilated by the organism's cells in an effort to produce energy, maintain life, or stimulate growth. The term food includes substance consumed to provide nutritional support for both the human and animal body.

“Vegetative propagation” or “clonal propagation” refers to propagation of plants from vegetative tissue, e.g. by propagating plants from cuttings or by in vitro propagation. In vitro propagation involves in vitro cell or tissue culture and regeneration of a whole plant from the in vitro culture. Clones (i.e. genetically identical vegetative propagations) of the original plant can thus be generated by in vitro culture. “Cell culture” or “tissue culture” refers to the in vitro culture of cells or tissues of a plant. “Regeneration” refers to the development of a plant from cell culture or tissue culture or vegetative propagation. “Non-propagating cell” refers to a cell which cannot be regenerated into a whole plant.

“Average” refers herein to the arithmetic mean.

It is understood that comparisons between different plant lines involves growing a number of plants of a line (or variety) (e.g. at least 5 plants, preferably at least 10 plants per line) under the same conditions as the plants of one or more control plant lines (preferably wild type plants) and the determination of differences, preferably statistically significant differences, between the plant lines when grown under the same environmental conditions. Preferably the plants are of the same line or variety.

### Methods the Introduction and Selection of a Specific Heritable Mutation in a Plant

The present invention provides a method for the introduction and selection of a specific heritable mutation in a plant comprising: (a) transfecting plant cells with exogenous DNA, wherein said exogenous DNA encodes an RNA-guided DNA endonuclease, guide RNA (gRNA) suitable for directing the RNA-guided DNA endonuclease to induce said specific heritable mutation and a selection marker; (b) regenerating plants from transfected cells to provide a plurality of T0 plants; (c) crossing the T0 plants with isogenic plants not comprising said exogenous DNA to provide a plurality of progeny plants; and (d) selecting one or more plants having the heritable mutation from the progeny plants.

By crossing T0 plants with wild type plants, it can be prevented that a very high number of TO plants need to be created and screened when the specific heritable mutation only occurs in low frequency. Without being bound to theory, it is believed that in at least a subset of T0 plants the DNA endonuclease/gRNA complex will be active. By crossing the T0 plants with wild type plants (i.e. plants not comprising an active construct), T0 plants having an active DNA endonuclease/gRNA complex will induce the desired mutation in the F1 generation (offspring of the cross between the T0 plants having an active construct and wild type plants). As a result thereof, it is possible to create a very high number of F1 plants, which are required to identify the specific heritable mutation of interest. Such a high number of F1 plants can be more easily obtained than a very high number of T0 plants. For instance, when the desired heritable mutation is an inversion in the genomic DNA of the target plant to reduce the genetic distance between two traits to zero, the most significant problem that has to be overcome is that the actual inversion occurs only in a very low frequency. It can even be very difficult to generate enough transgenic events to be able to identify a plant with the desired inversion. Instead, the present inventors surprisingly found that transfected plants with active constructs may be selected and to cross these plants with pollen from an isogenic wild-type plant having intact gRNA target sites. This isogenic wild-type pollen brings new template for the after fertilization. Consequently, a much higher amount of seedlings derived from seeds from these crosses can be screened easily by sampling cotyledons, DNA isolation and PCR-based screening. By using the method of the present invention, accordingly, many new individual plants can easily generated without the need of in vitro generation of de novo transfected or transformed individuals to identify a plant with the desired rare mutation. In fact, plants with an active CRISPR-Cas construct can be used as a “mutation inducer” in any pollen donor to make crosses. Only few back-crosses would be required to produce a donor genotype with the desired mutation.

The step of crossing the T0 plants with isogenic plants not comprising the exogenous DNA accordingly provides the active DNA endonuclease/gRNA complex with genomic DNA having intact gRNA target sites, thereby inducing the desired mutation by the active DNA endonuclease/gRNA. The process step of crossing the T0 plants with isogenic plants not comprising said exogenous DNA as comprised in the present invention as such thus represents a technical step that introduces a trait into the genome or modifies a trait in the genome of the plant produced. The introduction or modification of the trait in the process step of crossing the T0 plants with isogenic plants not comprising said exogenous DNA is not the result of the mixing of the genes of the plants chosen for the sexual crossing.

In one step of the method of the present invention, accordingly, plant cells are transfected with exogenous DNA. Means and methods for the introduction of exogenous DNA in plant cells are well-known in the art including, but not limited to, bacteria-mediated transformation (e.g. Agrobacterium-mediated transformation), virus-mediated gene transfer, particle-based transfection methods such as biolistics methods using particle bombardment, non-chemical methods such as electroporation, sonification and microinjection, and chemical methods such as liposome transfection; see e.g. Keith Lindsey and Wenbin Wei. In: Arabidopsis, A Practical Approach. Ed. Z. a. Wilson. Oxford Uni-versity Press, 2000. New York, USA. The introduction of exogenous DNA into plant cells can be performed in a plant cell culture, in a plant tissue culture or even in plant cells comprised in a whole plant. In the latter case, the transfected cells are isolated or separated from the non-transfected plant cells before regenerating plants from the transfected cells. Stable transformation by e.g. Agrobacterium is preferred when new mutation events need to be generated and identified in the transformed plant's offspring, which requires an active heritable DNA construct.

The exogenous DNA that is introduced into the plant cell in the method of the present invention encodes an RNA-guided DNA endonuclease, guide RNA (gRNA) suitable for directing the RNA-guided DNA endonuclease to induce said specific heritable mutation and a selection marker. The exogenous DNA that is introduced into the plant cell in the method of the present invention may encode further elements such as, but not limited to, a repair template. A repair template is a sequence of a nucleic acid molecule which functions as a template in the host cell's DNA repair process, particularly in the homology directed repair mechanism (HDR). It is however preferred that the exogenous DNA that is introduced into the plant cell in the context of the present invention does not comprise a repair template.

The exogenous DNA that is introduced into the plant cell in the method of the present invention accordingly encodes an RNA-guided DNA endonuclease. Various different RNA-guided DNA endonucleases that may be used in the method of the present invention have been meanwhile identified. In one aspect, the RNA-guided DNA endonuclease encoded by the exogenous DNA that is introduced into the plant cell in the method of the present invention is selected from the group consisting of Cas9, Cas12a, Cas12b, Cas12c, and Cas13. In one aspect, the RNA-guided DNA endonuclease encoded by the exogenous DNA that is introduced into the plant cell in the method of the present invention preferably is Cas12a (previously also named as Cpf1). Cas12a cleaves the DNA leaving sticky ends, which may lead to a more efficient subsequent DNA repair.

The exogenous DNA that is introduced into the plant cell in the method of the present invention further encodes a guide RNA (gRNA) suitable for directing the RNA-guided DNA endonuclease to induce the desired specific heritable mutation. The gRNA used in the context of this invention may consist of multiple RNAs forming a complex or may consist of a single gRNA (sgRNA), wherein different components of the gRNA are covalently connected, e.g. by one or more linkers. The design of the gRNA may vary greatly and is partially determined by the selection of the specific RNA-guided DNA endonuclease that is used in the method of the present invention. For instance, the selection of Cas9 as RNA-guided DNA endonuclease requires that the gRNA comprises a trans-activating crRNA (tracrRNA) and at least one CRISPR RNA (crRNA). Alternatively, the gRNA used in combination with Cas9 may also be a single guide RNA (sgRNA) comprising a tracrRNA and at least one crRNA combined in one RNA. In one aspect, accordingly, the RNA-guided DNA endonuclease in the process of the present invention is Cas9, wherein the gRNA comprises a tracrRNA and at least crRNA or wherein the gRNA is a sgRNA comprising a tracrRNA and at least one crRNA combined in one RNA. As a further example, the selection of Cas12a as RNA-guided DNA does not require that the gRNA comprises a tracrRNA. Instead, Cas12a requires that the gRNA comprises a T-rich protospacer-adjacent motif (PAM).

The method according to the present invention may be used to induce any heritable mutation in a plant. Such heritable mutation may be one or more mutations selected from the group consisting of a point mutation, a nonsense mutation, a missense mutation, a splice-site mutation, a frame shift mutation, a pre-mature stop codon mutation, a deletion, truncation, a replacement, an insertion, an inversion, duplication, and a translocation. The mutation may be in one or more selected from the group consisting of a coding region, a non-coding region, a regulatory sequence, such as promoter sequence or an enhancer sequence. The method according to the present invention is particularly useful for the introduction and selection of a heritable mutation that which is expected to occur only with a relatively low frequency. Such low frequency mutations for instance may be mutations that are obtained by non-homologous end joining (NHEJ). In a further aspect, accordingly, the present invention provides a method for the introduction and selection of a specific heritable mutation in a plant as described herein, wherein the specific heritable mutation is an inversion, duplication or a translocation. Such mutations are particularly useful when it is an objective to use the method of the present invention to obtain a plant that is not a transgenic plant.

The exogenous DNA that is introduced into the plant cell in the method of the present invention further encodes a selection marker which allows for selection of the plant cells in which the introduction of the exogenous DNA was successful and allows expression of the encoded RNA-guided DNA endonuclease and gRNA.

In a further step of the method of the present invention, plants are regenerated from the transfected cells to provide a plurality of T0 plants. Means and methods for regenerating plants from plant cells are well known in the art. For instance, the cell or tissue culture can be treated with shooting and/or rooting media to regenerate whole plants.

In one aspect of the present invention, the transfected cells from which the T0 plants are regenerated are selected based on the activity of the selection marker, wherein the selection marker is preferably selected from the group consisting of kanamycin resistance; chloramphenicol resistance, tetracycline resistance and ampicillin resistance.

In a further step of the method of the present invention, the T0 plants as obtained are crossed with isogenic plants not comprising said exogenous DNA to provide a plurality of progeny plants. It was surprisingly found that a much higher number of mutant plants can be obtained by crossing the T0 plants with isogenic plants not comprising the exogenous DNA. Consequently, a much higher number of seedlings derived from seeds from these crosses can be easily screened.

In a further step of the method of the present invention, one or more plants having the heritable mutation are selected from the progeny plants. Means and methods for screening for a specific mutation in large numbers of plants are well known in the art and may comprise screening at the DNA, RNA (or cDNA) or protein level using known methods, in order to detect the presence of the desired mutation(s). For instance, seedlings of the progeny plants obtained in the process of the present invention may be grown followed by taking of samples from a plant part such as the cotyledons. Subsequently, DNA may be isolated from the cotyledon samples followed by a PCR-based screening for the desired mutation(s).

DNA samples may be pooled before PRC-based screening to decrease the number of PCRs. For instance, 1000 DNA samples may be pooled per 100 pools each containing DNA from 10 plants. After a PCR-based screening of these 100 pools and identification of a mutation in one or more of these pools, in the next PCR individual DNA samples making up the identified pools may be screened to identify the DNA sample containing the mutation (One dimensional pooling).

Alternatively, for instance 900 DNA samples might be organized in a virtual two dimensional grit with 30 columns (x1 . . . x30) and 30 rows (y1 . . . y30) in which each sample has its own coordinate starting with x1y1 and ending with x30y30. Pooling the DNA from all columns leads to 30 x-pools and pooling DNA from all rows leads to 30 y-pools. PCR-based screening may be performed on those 30 x-pools and 30 y-pools and lead to the identification of a mutation in two pools; one in the x-pools and one in the y-pools. The numbers of the x-pool and y-pools lead to the identification of the specific DNA sample containing the mutation, e.g. x20y10.

Alternatively, for instance 1000 DNA samples might be organized in a virtual three-dimensional grit with 10 x-coordinates, 10 y-coordinates and 10 z-coordinates in which each DNA sample has its unique coordinate starting with x1, y1, z1 and ending with x10, y10, z10. Pooling the DNA samples in each dimension leads to 10 x-pools, 10 y-pools and 10 z-pools, each containing the DNA from 100 individuals, and each individual is represented in one pool of each dimension. PCR-based analysis of the 30 pools might lead to the identification of a mutation in one of the pools of each dimension e.g. x-pool 3, y-pool 5 and z-pool 4. This would lead to the identification of the DNA sample of one individual plant with the coordinate x3, y5, z4 (Tsai et al., 2011; https://doi.org/10.1104/pp. 110.169748).

PCR-based screening may be performed using primers that are specific for the expected or desired mutation. Primers may be designed to only amplify a DNA fragment when a specific inversion has taken place. For example, two primers designed in the same direction on the target DNA will not amplify a product. However, when an inversion occurs, leading to inversion of one of the primer binding sites, the new orientation of one primer relative to the other might allow the amplification of a specific DNA fragment that can be used as marker for that particular event.

Alternatively, a primer set may also be designed to only amplify a DNA fragment if a deletion has occurred. For example, the location of a forward orientated primer and a reverse primer might be too distant to amplify a product under chosen reaction conditions. A particular DNA polymerase might be able to transcribe 1 kbp per minute. In a PCR with 10 seconds extension time products of about 1 kbp or longer cannot be amplified. Mutations such as a deletion might lead to decreased physical distance of the primers and allow the production of an amplicon in a PCR with 10 seconds extension time, which would then be a marker for the deletion.

Alternatively, Digital PCR Systems such as droplet digital PCR (ddPCR) may be used to identify mutations in pooled DNA samples. Droplet Digital PCR fractionates a DNA sample in thousands of droplets. PCR amplification of the template subsequently occurs in each individual droplet, and counting the positive droplets gives precise, absolute target quantification. Digital PCR allows the detection and quantitation of rare sequences such as SNPs, allelic variants, edited DNA.

In a further aspect, accordingly, the present invention provides a method wherein the one or more plants having the heritable mutation are selecting by isolating genomic DNA from a part of the progeny plants and determining whether said genomic DNA comprises the heritable mutation.

Preferably, DNA sequencing is used to determine whether the genomic DNA of a plant obtained in the method of the present invention comprises the desired heritable mutation. Means and methods for DNA sequencing are well known in the art and include methods such as chain termination sequencing (Sanger sequencing), sequencing by synthesis (Illumina sequencing), Nanopore DNA sequencing, Polony sequencing, Ion semiconductor sequencing and DNA nanoball sequencing.

Alternatively, a SNP genotyping assay can be used to select a plant having the desired heritable mutation, particularly when it is sufficient to detect a single nucleotide difference (single nucleotide polymorphism, SNP) between a plant comprising the desired mutation and a plant which does not comprise the desired mutation. For example, the SNP can easily be detected using a KASP-assay (see world wide web at kpbioscience.co.uk) or other SNP genotyping assays. For developing a KASP-assay, for example 70 base pairs upstream and 70 base pairs downstream of the SNP can be selected and two allele-specific forward primers and one allele specific reverse primer can be designed. See e.g. Allen et al. 2011, Plant Biotechnology J. 9, 1086-1099, especially p 097-1098 for KASP-assay method. Equally other genotyping assays can be used such as a TaqMan SNP genotyping assay, a High-Resolution Melting (HRM) assay and SNP-genotyping arrays (e.g. Fluidigm, Illumina, etc.).

In a further aspect, the method of the present invention comprises a step wherein prior to crossing of the T0 plants with the wild type plants, T0 plants expressing the RNA-guided DNA endonuclease are selected to provide a one or more T0 plants having an active construct and wherein only said one or more T0 plants having an active construct are crossed with a wild type plant to provide the plurality of progeny plants. With the term “active construct” as used herein, it is meant that the DNA endonuclease/gRNA complex encoded by the exogenous DNA which was previously introduced is active, i.e. the DNA endonuclease/gRNA complex is capable of inducing the desired mutation in the F1 generation (offspring of the cross between the T0 plants having an active construct and wild type plants).

By selecting T0 plants having an active construct and crossing those with plants not having an active construct, screening of the offspring plants is more efficient. When only one or a relatively low number of T0 plants having an active construct are crossed with wild type plants (not having an active construct), a high number of different F1 plants can be generated and screened for the low frequency event. It is much more efficient to create a very high number of F1 plants which may have the specific heritable mutation when the T0 plants wherein the DNA endonuclease/gRNA complex is not active are removed from the population that is crossed with the wild type plants.

In one aspect, the method of the present invention comprises a step wherein the selected TO plants expressing the RNA-guided DNA endonuclease are T0 plants in which RNA of said RNA-guided DNA endonuclease is detected. One indirect method to identify T0 plants with active CRISP-Cas construct is the presence of mutations caused by the active construct. For example, a CRISPR-Cas construct might encode two or more guide RNAs that target the flanks of a genomic DNA fragment to be inverted. The desired inversion might be a seldom occurring event and might not be present in the T0 generation. However, mutations leading to small deletions might occur at the gRNA target site depending on the activity of the construct. The presence of these mutations in heterozygous state demonstrate an active construct. Mutations in homozygous state might even be a better proof that the CRISPR-Cas construct is active.

In a further aspect, the method of the present invention comprises a step wherein the progeny plants are selfed followed by selecting one or more plants having the heritable mutation from the progeny plants.

By first selfing the F1 progeny plants to obtain a F2 progeny population and selecting for plants having the heritable mutation from said F2 progeny population, plants homozygous for the presence of the active construct are obtained. F1 progeny plants comprising the active construct are always heterozygous for the active construct. Alternatively, T0 plants may be selfed to obtain homozygous T1 plants, which can be crossed with isogenic plants not comprising the exogenous DNA to provide a plurality of progeny plants. Accordingly, the method of the present invention may comprise a step wherein the T0 plants are selfed to obtain T1 plants, which are crossed with isogenic plants not comprising the exogenous DNA to provide a plurality of progeny plants.

In a further aspect, the method of the present invention further comprises that a subset of the plurality of T0 plants (as obtained in method step (a) as described herein above) are subjected to at least one selfing step and wherein the offspring plants obtained by said at least one selfing step are pooled (i.e. combined) with the progeny plants (as obtained in method step (c) as described herein above) from which one or more plants having the heritable mutation are selected. Accordingly, part of the T0 plants which are regenerated from the transfected cells are subjected to at least one selfing step instead of crossing with isogenic plants not comprising the exogenous DNA. The plants obtained from said at least one selfing step are pooled with the progeny plants obtained by the crossing of the T0 plants with isogenic plants not comprising said exogenous DNA, which is followed by the step of selecting one or more plants having the heritable mutation.

In a further aspect, the method of the present invention further comprises that the selected one or more plants having the heritable mutation are backcrossed to obtain a progeny plant which comprises the specific heritable mutation and which does not comprise any exogenous DNA.

The following non-limiting Examples describe how one can efficiently obtain mutant tomato plants using the method according to the present invention, wherein said tomato plants comprise in their genome at least one chromosome comprising a mutant allele of the wild type male sterility 10 (MS10) gene and a mutant allele of the wild type anthocyanin absent (AA) gene wherein in said plant meiotic recombination is suppressed between said mutant allele of the wild type MS10 gene and said mutant allele of the wild type AA gene. Unless stated otherwise in the Examples, all recombinant DNA techniques are carried out according to standard protocols as described in Sambrook et al. (1989) Molecular Cloning: A Laboratory Manual, Second Edition, Cold Spring Harbor Laboratory Press, and Sambrook and Russell (2001) Molecular Cloning: A Laboratory Manual, Third Edition, Cold Spring Harbor Laboratory Press, NY; and in Volumes 1 and 2 of Ausubel et al. (1994) Current Protocols in Molecular Biology, Current Protocols, USA. Standard materials and methods for plant molecular work are described in Plant Molecular Biology Labfax (1993) by R. D. D. Croy, jointly published by BIOS Scientific Publications Ltd (UK) and Blackwell Scientific Publications, UK. Standard breeding methods are described in ‘Principles of Plant breeding’, Second Edition, Robert W. Allard (ISBN 0-471-02309-4).

### EXAMPLES

### Example 1

### Designing and Cloning of the Constructs

For design of gRNAs for MS, we used the first exon of MS. For design of gRNAs for AA, we used the first and second exon of this gene. For each gene we designed two gRNAs (Table 1), using the software CRISPOR (http://crispor.tefor.net).

TABLE 1
gRNAs for making double strands DNA breaks
in the male sterility gene MS and the
anthocyanin absent gene AA, both on Ch02.

| Targeted | | | Position |
|---|---|---|---|
| gene | name | gRNA | SL3.0ch02 |
| MS | gMS1 | GGCGTCAAAAACTTAGCGAA | 44796492 |
| | | AGG (SEQ ID NO: 5) | |
| MS | gMS2 | ATACAAATCCAAGAACCTTA | 44796528 |
| | | AGG (SEQ ID NO: 6) | |
| AA | gAA1 | GAAAGTGTATGGTTCAGCAA | 45896386 |
| | | TGG (SEQ ID NO: 7) | |
| AA | gAA2 | CAATGGCTGCATGTCCACAA | 45896403 |
| | | AGG (SEQ ID NO: 8) | |

Two CRISPR-Cas constructs were made, combining one gRNA for AA with one for MS.

Construct 1 contained gMS1 and gAA1, Construct 2 harboured gMS2 and gAA2 (Table 1).

The CRISPR-Cas constructs were built, by means of cloning using the Golden Gate MoClo Toolkit (https://www.addgene.org/kits/marillonnet-moclo/). The designed gRNAs were ordered as oligos and each one was inserted separately behind the U6-26 promotor from Arabidopsis thaliana (pICSL90002 plasmid, https://www.addgene.org/68261/). The gRNAs with promotor were combined with the Arabidopsis codon-optimized SpCas9 sequence under the Petroselinum crispum Ubiquitin4-2 promoter and NOS terminator (pDe-CAS9, https://www.addgene.org/61433/). We included in the CRISPR-Cas construct a fluorescence GFP gene (p35S-fGFP-ter35S), driven by CaMV-35S promotor and terminator, for estimation of the proportion of successfully transfected protoplasts.

### Plasmid Isolation, Harboring CRISPR-Cas Construct 1 or 2

The plasmids harbouring the two CRISPR-Cas constructs were propagated in Escherichia coli (25 ml LB) and isolated, using the QIAGEN plasmid midi kit (Cat No./ID: 12143), as described in the manual.

The plasmid DNA was eluted in 200 μl EB buffer and quantified using the Nanodrop One (Thermofisher). For the transfection, 10 ug plasmid was pipetted in a 2 ml tube, and water was added till the volume reached 20 μl.

### Plant Material

Tomato seeds of the cultivar Moneymaker were sterilized with 1% hypochlorite and 0.01% Tween20. Subsequently, the seeds were washed 3 times with sterile water and placed on germination medium in pots. The germination medium consisted of 2.2 gram MS medium (Duchefa) with added MS vitamins (Duchefa), and 5 gram sucrose per liter. The pH was adjusted to 5.8. The pots were placed at a 16 hours light and 8 hours dark regime at 24° C.

The young seedlings were transplanted to pots with air filter, two seedlings per pot. The pots contained 4.4 g/l MS medium with vitamins (Duchefa), 30 gram sucrose per liter, and the pH was adjusted to 5.8. Plants were grown for 4 weeks under 16 hour light and 8 hour darkness at 24° C., till several fully developed leaves were present.

### Transfections

For each transfection, healthy expanded leaves were harvested from two pots, cut into small strips in enzyme digestion buffer, and incubated overnight. Protoplast were filtered, washed, and treated with PEG and plasmid for transfection using standard methods. Subsequently, the protoplasts were washed, put in recovery medium, and incubated in the dark at 24° C. for 24 hours. Transfection efficiency was measured by estimating the proportion of protoplasts that showed green fluorescence because of the presence of the GFP gene. For every transfection reaction there were approximately 200,000 protoplasts in an end volume of 150 μl. This resembles a concentration of approximately 1,300 protoplasts per μl.

### Checking Presence of Induced Inversions

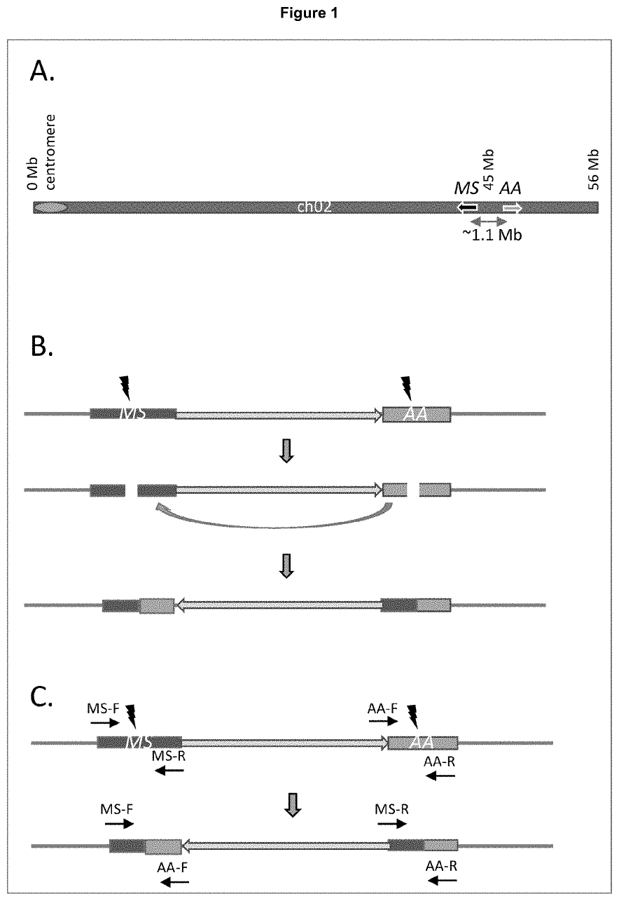

For checking for presence of induced mutations, we used targeted PCRs, as shown in FIGS. 1B and C. Forward and reverse primers were designed around the MS and AA guides, using the reference genome for sequences flanking the gRNA loci, and primer design software Primer3 (http://bioinfo.ut.ee/primer3-0.4.0/). The primers pairs are (AA-F) 5′-TGGTTGCTGCTCATCTTCAC-3′ (SEQ ID NO: 9) with (AA-R) 5′-GCAAAGCCACCTTCATTCAT-3′ (SEQ ID NO: 10) and (MS-F) 5′-TAGGGGATTTTCATGCTGGT-3′ (SEQ ID NO: 11) with (MS-R) 5′-GCCAAAAATGAGTCCTTCCA-3′ (SEQ ID NO: 12).

The transfected protoplasts were processed in the following way: Per sample, 2 μl of the isolated protoplasts were diluted 1:1 with a 20 mM KOH, 1% caseine solution, boiled for 5 minutes, and put on ice for 5 minutes.

The left-hand side and the ‘right-hand side’ of the induced inversions were amplified by means of PCR, using Phire polymerase (Thermofisher). For the left end, the forward MS primer (MS-F) and forward AA primer (AA-F) were used, while for the right-hand side we used the reverse MS-R and reverse AA-R primers) (FIG. 1C). The reaction mix contained 5 μl 5× Buffer, 1 μl 5 mM DNTP, 1.25 μl 10 pmol/μl F primer, 10 pmol/μl R primer, 0.35 μl Phire polymerase, 4 μl protoplast solution (˜2700 protoplasts), and 12.5 μl water. A water control was also included. Eighty PCR cycles (10 sec at 98° C., 20 sec at 61.5° C., and 40 sec extension time at 72° C.) were performed to detect also rarely occurring events. As a negative control, non-transfected protoplasts were used. PCR products were brought on agarose gel to visualize them.

qPCR

To estimate the frequency of the inversion events, a qPCR was performed on the processed protoplast samples. This was done with the iQ SYBR Green Supermix (Biorad) on the qPCR machine (Biorad). Reaction conditions were as follows: 12.5 μl iQ SYBR Green Supermix, 1.25 μl 10 pmol/μl F primer, 10 pmol/μl R primer, 2 μl protoplast solution (˜1300 protoplasts), water till 25 μl.

As reference the Actin gene from tomato was included, using as forward and reverse primer sequences 5′-ACTGTCCCTATCTATGAAGGTTATGC-3′ (SEQ ID NO: 13) and 5′-GAAACAGACAGGACACTCGCACT-3′(SEQ ID NO: 14), respectively.

### Plant Transformation

Transgenic plants (TO) containing the CRISPR-Cas were generated by Agrobacterium tumefaciens-mediated transformation. The following transformation method may accordingly be used. Cotyledons of 10-day-old seedlings are incubated in 8 ml Agrobacterium cells suspended in 2% MSO (liquid MS medium, containing 100 mg L⁻¹ myo-inositol, 400 μg L⁻¹ thiamine HCl and 20 g L⁻¹ sucrose) to an attenuance at 600 nm of 0.5. After 30 min, the cotyledons are blotted dry on sterile filter paper and placed on MS culture medium containing 1× Nitsch and Nitsch vitamin mixture, 3% w/v sucrose, 1 mg L⁻¹ NAA, 1 mg L⁻¹ 6-benzylaminopurine and 0.7% w/v agar, pH 5.7. After 2 days of co-cultivation, the cotyledons are washed in liquid MS medium with 200 mg L⁻¹ carbenicillin and transferred to shoot-inducing MS culture medium containing 1× Nitsch and Nitsch vitamin mixture, 3% w/v sucrose, 2 mg L⁻¹ zeatin, 200 mg L⁻¹ carbenicillin, 0.7% w/v agar, pH 5.7 and 100 mg L⁻¹ kanamycin for selection. Cotyledons that start to develop callus are transferred to fresh culture medium, containing half of the zeatin concentration and 1 mg L⁻¹ GA3. The cotyledons are transferred to fresh medium every 2 weeks. When initial calli formed, shoot primordia are excised and transferred to shoot-elongation MS culture medium, which is germination medium containing 200 mg L⁻¹ carbenicillin and 100 mg L⁻¹ kanamycin. Elongated shoots of 2-4 cm were excised from the callus and transferred to rooting MS culture medium (1× Nitsch and Nitsch vitamin mixture, 1.5% w/v sucrose, 5 mg L⁻¹ IAA, 200 mg L⁻¹ carbenicillin, 50 mg L⁻¹ kanamycin and 0.7% w/v agar, pH 5.7). Rooted (TO) plantlets are transferred to soil for further analysis. Media components and antibiotics are obtained from Duchefa Biochemie.

### Selection of T0 Transgenic Plant Containing an Active the CRISPR-Cas Construct

The activity of the CRISPR-Cas construct is dependent on the location of the integration in the genome. To identify T0 plants with an active construct plants containing mutations were identified by sequencing the targeted regions in the MS and AA genes in the T0 plants. These domains were amplified by means of PCR, using Phire polymerase (Thermofisher). Primer set specific for either the targeted region in the MS or the AA genes were used. The reaction mix contained 5 μl 5x Buffer, 1 μl 5 mM DNTP, 1.25 μl 10 pmol/μl forward primer, 10 pmol/μl reverse primer, 0.35 μl Phire polymerase, 4 μl genomic DNA solution (4 ng), and 12.5 μl water. Thirty PCR cycles (10 sec at 98° C., 20 sec at 60° C., and 5 sec extension time at 72° C.) were performed to amplify the target regions. PCR products were sequenced by a service provider and the presence of mutations determined using a computer program for DNA sequence analysis.

Self-Pollination and Crossing T0 Plants with Plants not Comprising Said Exogenous DNA to Provide a Plurality of Progeny Plants

Selected T0 plants with an active CRISPR-Cas construct were grown and flowers were allowed to self-pollination to produce fruits with T1 seeds. Fruits were grown an T1 seeds were collected.

Alternatively, selected T0 plants with an active CRISPR-Cas construct were grown and flowers were emasculated before anther dehiscence to prevent self-pollination. Pollen, isolated from a wild type plant were collected and used to pollinate selected T0 plants, fruits were grown and F1 seeds were collected. F1 plants containing an active CRISPR-Cas construct can be grown to produce F2 seeds.

T1, F1 and F2 seeds were germinated and genomic DNA isolated from first leaves. PCRs were designed to detect desired mutations, inversions or other structural variations. By using a pooling strategy (e.g. Tsai et al., 2011; https://doi.org/10.1104/pp. 110.169748) large number of seedlings can be analyzed with only limited number of PCRs.

### Example 2

CRISPR-Cas9 constructs were built as described in Example 1. Three CRISPR-Cas9 constructs were made, combining one gRNA for AA with one for MS. Construct 1 contained gMS1 and gAA1, construct 3 gMS3 and gAA3, and construct 4 gMS4 and gAA4.

TABLE 2
gRNA target sites for making double-strand DNA
breaks in the male sterility gene MS and the
anthocyanin absent gene AA, both on Ch02 of
tomato. The [symbol] sign is placed at the location
where the double-strand break is expected to
take place. The PAM site is displayed in italics.

| Targeted | | | Position |
|---|---|---|---|
| gene | name | gRNA | SL3.0ch02* |
| MS | gMS1 | GGCGTCAAAAACT | 44796509-44796487 |
| | | TAGC [symbol] GAA AGG | |
| | | (SEQ ID NO: 5) | |
| MS | gMS3 | AACTCTGAAGAAA | 44796612-44796590 |
| | | GGGA [symbol] AGT AGG | |
| | | (SEQ ID NO: 15) | |
| MS | gMS4 | ATTCAAACAACTC | 44796620-44796598 |
| | | TGAA [symbol] GAA AGG | |
| | | (SEQ ID NO: 16) | |
| AA | gAA1 | GAAAGTGTATGGT | 45896370-45896392 |
| | | TCAG [symbol] CAA TGG | |
| | | (SEQ ID NO: 7) | |
| AA | gAA3 | AATGGCTGCATGT | 45896388-45896410 |
| | | CCAC [symbol] AAA GGG | |
| | | (SEQ ID NO: 17) | |
| AA | gAA4 | ATGGTTTGTCTTA | 45896413-45896435 |
| | | TAGA [symbol] ATT GGG | |
| | | (SEQ ID NO: 18) | |

*Solanum lycopersicum cultivar Heinz 1706 chromosome 2, SL3.0.

The constructs were propagated in Escherichia coli, and plasmids were isolated and purified for transfection of tomato protoplasts. Protoplasts were isolated from in vitro grown plant leaves of ‘Moneymaker’. After transfection and incubation, the cell cultures were screened for inversions by PCR. To do that a part of the transfected protoplasts (2.700 cells) were transferred to a PCR tube and denatured in KOH as described in Example 1.

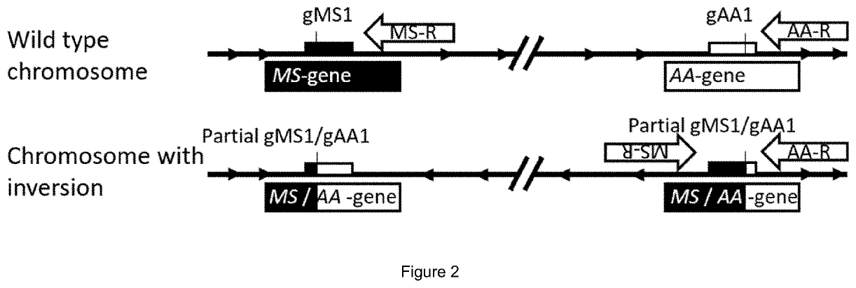

A PCR was performed on the cell lysate using primer combinations pairs AA-F with MS-F or AA-R with MS-R in separate PCRs. The annealing strand of one pair of primers is the same, preventing amplification on wild type DNA, and allowing amplification only in case of an induces inversion (FIG. 2). In addition, the distance between the primers of one pair is ˜1.1 Mbp on the wild type genome, which too large to amplify a PCR product.

TABLE 3
PCR primers used to amplify the borders of
the inversion

| Primer | | Position |
|---|---|---|
| name | Primer sequence | SL3.0ch02* |
| AA-F | 5′-TGGTTGCTGCTCATCTTCAC-3′ | 43326933-43326952 |
| | (SEQ ID NO: 9) | |
| MS-F | 5′-TAGGGGATTTTCATGCTGGT-3′ | 42223166-42223185 |
| | (SEQ ID NO: 11) | |
| AA-R | 5′-GCAAAGCCACCTTCATTCAT-3′ | 43328249-43328230 |
| | (SEQ ID NO: 10) | |
| MS-R | 5′-GCCAAAAATGAGTCCTTCCA-3′ | 42224500-42224481 |
| | (SEQ ID NO: 12) | |

*Solanum lycopersicum cultivar Heinz 1706 chromosome 2, SL3.0.

However, an inversion of the region between the gRNA targets, would bring together the primer sites in an orientation that would make amplification of a PCR product possible, including the border of the inversion as depicted in FIG. 2.

PCR products from protoplasts transfected with construct 1, 3, and 4 were separated on an agarose gel and fragments with the expected size were excised from gel and the DNA was Sanger sequenced. It was surprising that in most PCRs DNA fragments the expected size were generated, which would mean that inversions take place in one or more protoplasts per reaction. Considering that one reaction contains the DNA from about 2700 protoplasts, and that the transfection efficiency was about 70% (appearing from fluorescence of the GFP gene, present in the constructs too; data not shown), that would mean that the inversion frequency is >1/(0.7*2700) cells. The expected PCR amplicon sizes were based on the location of the primer and gRNA binding sites on the genome, which is about 1.3 kb. The primer and gRNAs binding site locations and their sequences are depicted in tables 2 and 3.

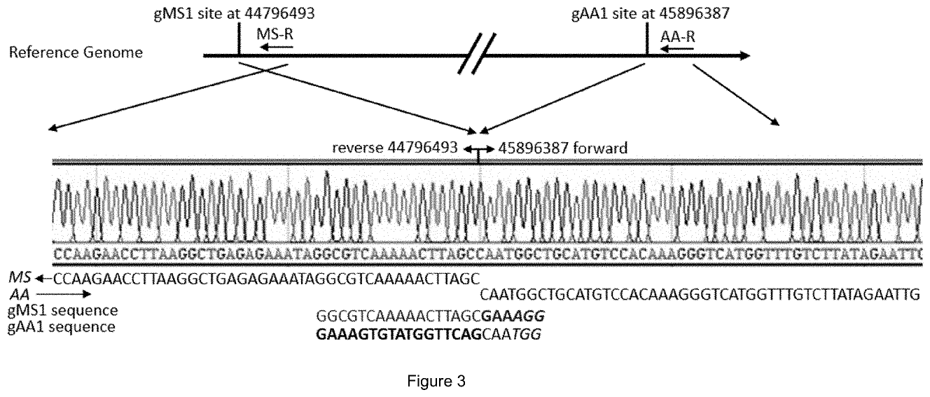

FIG. 3 shows a part of the PCR product's DNA sequence generated with the primers MS-R and AA-R, and genomic DNA from a protoplast culture transfected with construct 1, harboring the gRNAs gMS1 and gAA1. The sequence is from the downstream right end of the inversion. The alignment in the lower part of the figure shows that the DNA had been cleaved in the gMS1 binding site, and that the upstream part has been linked to the gAA1 binding site. Apparently, the Cas enzyme had generated double-strand breaks (DSBs) at both gRNA binding sites, leading to the inversion of the ˜1.1 Mbp chromosome fragment in between. The DSB were generated at exactly the predicted locations in the gRNA binding sites, which is between three and four basepairs upstream the PAM site. The ligation of the ends of the inverted chromosome fragment had been made without any additional sequence modification.

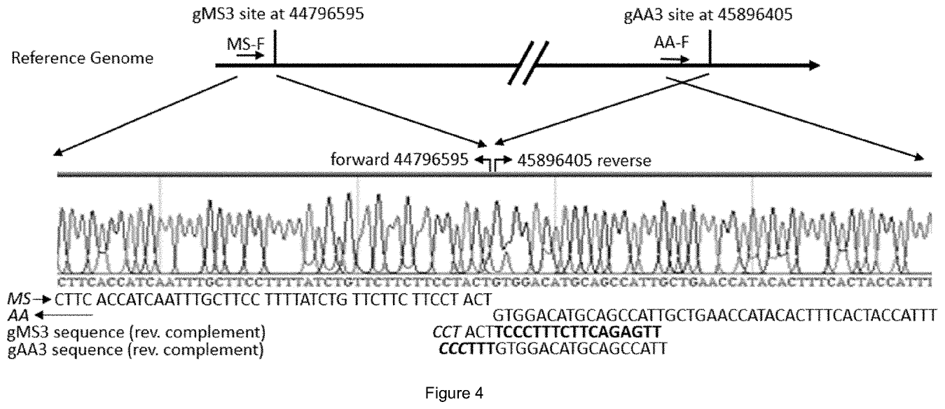

FIG. 4 shows part of the PCR product's DNA sequence generated with primers MS-F and AA-F and genomic DNA from protoplasts transfected with construct 3. The sequence is from the upstream left end of the inversion. The alignment in the lower part of the figure shows that the DNA had been cleaved at the gMS3 binding site and that the upstream part has been linked to the gAA3 binding site.

The Cas9 enzyme also here generated double-strand breaks (DSBs) at both gRNA binding sites, leading to the inversion of the ˜1.1 Mbp chromosome fragment in between. The DSBs were generated at exactly the predicted locations in the gRNA binding sites, and the ligation of the ends of the inverted chromosome fragment had been made without any additional sequence modification.

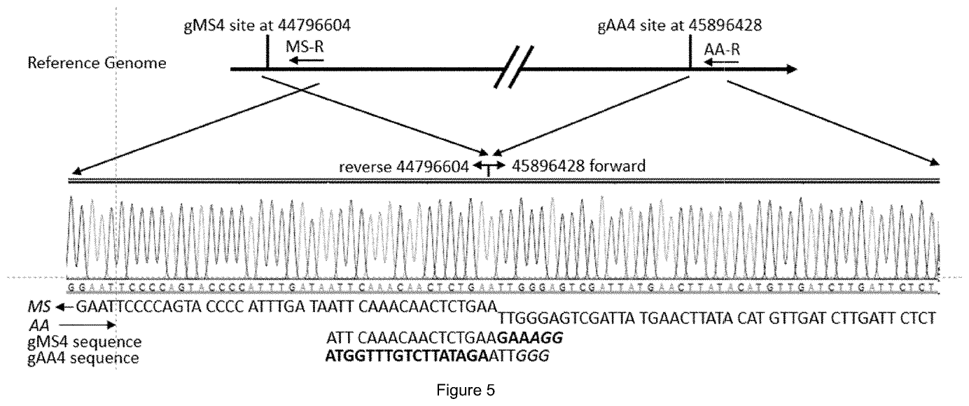

FIG. 5 shows part of the PCR product's DNA sequence generated with primers MS-R and AA-R and genomic DNA from protoplasts transfected with construct 4. The sequence is the downstream right end of the inversion. The alignment in the lower part of the figure shows that the DNA had been cleaved at the location of the gMS4 binding site, and that it has been linked to the location of gAA4 binding site. The Cas enzyme had also here generated the DSBs at both sides of the induced inversion at exactly the predicted location in the gRNA binding sites, and the ligation of the ends has been made with a deletion of one adenosine at the ligation site.

The three sequences in the FIGS. 3 to 5 show that the gRNAs target the Cas9 to the predicted positions and that DNA cleavage takes place at the expected position. In addition, the sequenced transitions demonstrate that DSBs had been generated at two locations on the chromosome and that in some cases the chromosome fragment in-between had been inversed after repair. The sequences of the ligated ends (the transition) demonstrate that the DSBs were generated at the predicted positions.

Example 2 accordingly shows that inversions can be detected by amplification of the borders from the inversion in most of the PCRs containing the DNA from ˜2700 protoplasts. As explained above this would mean that the inversion takes place in about 1 of 2700*0.7(˜transfection efficiency)=—1900 protoplasts. To obtain a plant with the desired mutation would require a large number (>1900) regenerated shoots to have a chance on finding one with the desired mutation. In many species, including tomato, regeneration of shoots from protoplasts is technically very difficult, and has in some species never successfully been applied.

To solve this technical problem, the present invention provides a seed-based screening approach to identify such mutation. In general, the CRISPR-Cas construct causes double strand breaks in the DNA, that in the majority of events are repaired. The repair system often modifies the guide-RNA (gRNA) binding site, meaning that after repair the binding site for the gRNA is gone and no new double strand breaks can be generated. However, when a plant containing an active CRISPR-Cas construct is crossed with a wild type plant, then at least half of the seeds produced will contain an active CRISPR-Cas construct in addition to wild type DNA with unmodified gRNA binding sites. This means that each seed from such plant provides a new chance of inducing and identification of the seldom occurring mutation event. Thus, if an event, e.g. an inversion, occurs in 1 per 1900 events, then screening a multiplicity of this number of seedlings can be done to identify the desired mutation. The screening could be a PCR with border specific primers as describes above for protoplasts. To reduce the number of individual PCRs, a pooling strategy, one-, two- or three-dimensional may be designed.

The herein described method accordingly for the first time enables the provision of tomato plants comprising three desirable traits at the same time:

1. Knocking out MS, leading to male sterility in homozygous plants, facilitating production of hybrid seeds;
2. Knocking out AA, leading to absence of anthocyanin, and therefore loss of purple hypocotyl color.
3. Genetic linkage of these two traits, because of suppression of meiotic recombination between the mutant alleles ms and aa caused by the inversion, and therefore loss of homology of the sequence between the two genes.

1. A method for the introduction and selection of a specific heritable mutation in a plant comprising:
(a) transfecting plant cells with exogenous DNA, wherein said exogenous DNA encodes an RNA-guided DNA endonuclease, guide RNA (gRNA) suitable for directing the RNA-guided DNA endonuclease to induce said specific heritable mutation and a selection marker;
(b) regenerating plants from transfected cells to provide a plurality of T0 plants;
(c) crossing the T0 plants with isogenic plants not comprising said exogenous DNA to provide a plurality of progeny plants; and
(d) selecting one or more plants having the heritable mutation from the progeny plants.

2. The method of claim 1, wherein prior to crossing of the TO plants with the isogenic plants, T0 plants expressing the RNA-guided DNA endonuclease are selected to provide a one or more T0 plants having an active construct and wherein only said one or more T0 plants having an active construct are crossed with a wild type plant to provide the plurality of progeny plants.

3. The method of claim 2, wherein the selected T0 plants expressing the RNA-guided DNA endonuclease are T0 plants in which RNA of said RNA-guided DNA endonuclease is detected.

4. The method of claim 1, wherein the progeny plants are selfed followed by selecting one or more plants having the heritable mutation from the progeny plants.

5. The method of claim 1, wherein a subset of the plurality of T0 plants are subjected to at least one selfing step and wherein the offspring plants obtained by said at least one selfing step are pooled with the progeny plants from which one or more plants having the heritable mutation are selected.

6. The method of claim 1, wherein the specific heritable mutation is an inversion, duplication or a translocation.

7. The method of claim 1, wherein the RNA-guided DNA endonuclease is selected from the group consisting of Cas9, Cas12a, Cas12b, Cas12c, and Cas13.

8. The method of claim 7, wherein the RNA-guided DNA endonuclease is Cas9 and wherein:
the gRNA comprises a trans-activating crRNA (tracrRNA) and at least one CRISPR RNA (crRNA); or
the gRNA is a single guide RNA (sgRNA) comprising a tracrRNA and at least one crRNA combined in one RNA.

9. The method of claim 1, wherein the selected one or more plants having the heritable mutation are backcrossed to obtain a progeny plant which comprises the specific heritable mutation and which does not comprise any exogenous DNA.

10. The method of claim 1, wherein the transfected cells from which the T0 plants are regenerated are selected based on the activity of the selection marker.

11. The method of claim 1, wherein the one or more plants having the heritable mutation are selecting by isolating genomic DNA from a part of the progeny plants and determining whether said genomic DNA comprises the heritable mutation.

12. The method of claim 10, wherein the selection marker is kanamycin resistance or ampicillin resistance.

13. The method of claim 11, wherein the part of the progeny plants is a cotyledon.
